(19) United States
(12) Patent Application Publication
(10) Pub. No.: US 2025/0242112 A1
(43) Pub. Date: Jul. 31, 2025

(54) DRIVE MECHANISM FOR A DRUG DELIVERY DEVICE

(71) Applicant: Sanofi, PARIS (FR)

(72) Inventors: Alexander Allerdings, Frankfurt am Main (DE); Michael Schabbach, Frankfurt am Main (DE); Meinolf Werner, Worms (DE)

(21) Appl. No.: 18/844,097

(22) PCT Filed: Mar. 9, 2023
(86) PCT No.: PCT/EP2023/055992

§ 371 Date: Sep. 5, 2024

(30) Foreign Application Priority Data
Mar. 11, 2022 (EP) 22315059.0

(51) Int. Cl. A61M 5/315

(52) U.S. Cl. CPC A61M 5/31571; A61M 5/31578; A61M 2005/31588

(57) ABSTRACT

The present disclosure relates to a drive mechanism for moving a stopper inside a longitudinally extending barrel of a medicament container. The drive mechanism includes a drive member movable parallel to the longitudinal extent of the barrel, and a magnetic coupling comprising an outer coupling member and an inner coupling member, wherein the outer coupling member, which is mechanically engaged with the drive member, is configured to move along an outside surface of the barrel and wherein the inner coupling member, which is mechanically engagable with the stopper, is configured for insertion into the barrel. The at least one of the outer coupling member and the inner coupling member includes a first magnet and a second magnet separated from each other in the longitudinal direction and being configured to magnetically couple with a third magnet of the other one of the outer coupling member and the inner coupling member.

### CROSS REFERENCE TO RELATED APPLICATIONS

The present application is the national stage entry of International Patent Application No. PCT/EP2023/055992, filed on Mar. 9, 2023, and claims priority to Application No. EP 22315059.0, filed on Mar. 11, 2022, the disclosures of which are incorporated herein by reference.

### TECHNICAL FIELD

The present disclosure relates to the field of drug delivery devices, in particular for delivery of liquid medicaments, e.g. by way of infusion or injection. In one aspect the present disclosure relates to a drive mechanism for moving a stopper of a cartridge filled with a liquid medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

With drug delivery device or injection devices a liquid medicament is typically provided in a medicament container such as a cartridge, a syringe or capsule. Such medicament containers are typically closed or sealed towards a proximal direction by way of a stopper movably disposed in a barrel of such medicament containers. The liquid medicament can be expelled or dispensed from the medicament container by displacing, e.g. urging the stopper in distal direction relative to the barrel of the container. The distal outlet of the medicament containers may be in fluid connection with an injection needle or infusion line.

For introducing or transferring a driving force into or onto the stopper there exist mechanically implemented drive mechanisms, e.g. comprising an elongated piston rod in longitudinal abutment with a proximal end face of the stopper. Drive mechanisms known so far are typically operable to advance the piston rod in distal direction so as to exert a dispensing pressure onto the stopper of the medicament container.

With increasing digitalization and with the availability of electrically implemented drive mechanisms, i.e. drive mechanisms, wherein a force for moving the stopper in a distal direction is provided or generated by an electric or electromechanical drive, there is an increasing demand for new solutions and approaches of how to provide a mechanical coupling between the driver or drive member of the drive mechanism with the stopper of the medicament container, e.g. with the stopper of a cartridge. Moreover, it is also desirable to miniaturize such drive mechanisms and drug delivery devices. The drive mechanism, in particular the engagement or interface between a component of a drive mechanism and the stopper of the medicament container to be moved by the drive mechanism should be rather failure safe, robust and efficient in terms of a longitudinal force transfer.

### SUMMARY

In one aspect the present disclosure relates to a drive mechanism for moving a stopper of a medicament container, in particular for moving a stopper of a cartridge. The stopper is movably disposed inside a longitudinally extending barrel of the medicament container or cartridge. The drive mechanism comprises a drive member movable along a longitudinal direction parallel to the longitudinal extent of the barrel. The drive mechanism further comprises a magnetic coupling. The magnetic coupling comprises an outer coupling member and an inner coupling member. The outer coupling member is mechanically engaged with the drive member. It is configured to move along an outside surface of the barrel.

The inner coupling member is mechanically engageable with the stopper. With some examples the inner coupling member is temporarily or permanently mechanically engaged, e.g. mechanically fastened or fixed to the stopper. The inner coupling member is sized and configured for insertion into the barrel. Moreover, at least one of the outer coupling member and the inner coupling member comprises a first magnet and a second magnet. The first magnet and the second magnet are separated from each other in the longitudinal direction. The first magnet and the second magnet are further configured to magnetically couple or to magnetically interact with a third magnet of the other one of the outer coupling member and the inner coupling member.

By way of a first magnet and a second magnet separated from each other in the longitudinal direction a rather precise, strong, and well-defined magnetic coupling can be provided between the outer coupling member and the inner coupling member.

The outer coupling member, which is mechanically connected or fixed to the drive member of the drive mechanism, is movable along the outside surface of the barrel. Typically, and for expelling of the medicament from the barrel the drive member is movable along the longitudinal direction towards a distal end of the barrel of the medicament container. Also, the inner coupling member is located at or near the stopper of the cartridge. When mechanically engaged, i.e. when connected to or when in abutment with the stopper and when the outer coupling member gets in the vicinity to the inner coupling member the magnetic interaction between the outer coupling member and the inner coupling member will start to develop.

As the outer coupling member approaches the inner coupling member as seen in longitudinal direction, the magnetic interaction will increase successively until a mechanical force effect transferred via the magnetic coupling between the outer coupling member and the inner coupling member is effective and sufficient to move the inner coupling member and hence the stopper of the medicament container along the same direction as the outer coupling member is moved by the drive member.

With two magnets separated along the direction of movement, e.g. separated along the longitudinal direction, the magnetic interaction with the third magnet can be improved. Mechanical forces or magnetic forces between the arrangement of first and second magnets with regard to the third magnet can be increased. In this way a rather precise and strong magnetic and hence quasi-mechanical coupling can be provided between the outer coupling member and the inner coupling member.

With some examples it is the outer coupling member that comprises the first and the second magnets. Then, it is the inner coupling member that comprises the third magnet. With other examples it is the inner coupling member that comprises the first and the second magnets, whereas the outer coupling member comprises the third magnet. Providing of the first and the second magnet on one of the outer coupling member and the inner coupling member and providing of only one third magnet on the other one of the outer coupling member and the inner coupling member is generally sufficient to obtain the presently intended or desired magnetic interaction between the outer coupling member and the inner coupling member.

In effect, the magnetic field of the first practice interacts with the magnetic field of the second that. This way, a resulting magnetic field of first and second magnet is provided, which is interacting with the magnetic field of the third magnet.

According to a further example the first magnet and the second magnet are separated by a gap of predefined longitudinal size. The gap may comprise a circumferential gap. It may comprise a free annular space located between the first magnet and the second magnet. The gap, which is located longitudinally between the first magnet and the second magnet may be provided with a spacer. The spacer may entirely or at least partially fill the gap so as to keep the first magnet and the second magnet in a well-defined and non-overlapping longitudinal distance from each other. By way of a spacer or mount, the relative position of the first magnet and the second magnet can be precisely controlled.

With some examples the gap between the first magnet and a second magnet is implemented as an air gap. Hence, the gap between the first magnet and the second magnet is a void or empty space. It may not be filled or occupied by a material. With other examples, there may be arranged a component or an object inside the gap between the first magnet and the second magnet. The component or object located inside or protruding inside the gap may be of non-ferromagnetic and non-paramagnetic type. In particular, the component or object located inside or protruding into the gap between the first and the second magnets may be of diamagnetic type. Effectively, the space or gap between the first magnet and the second magnet may be magnetically inert.

According to a further example at least one of the first magnet and the second magnet comprises a magnetic pole of a first type and another magnetic pole of a second type. The magnetic poles of the first and second types are separated in longitudinal direction. Hence, a north pole of e.g. the third magnet may face towards a proximal direction and the corresponding south pole of the respective magnet may face in distal direction. Typically, the magnetic orientation of the first magnet and the second magnet is substantially parallel. Hence, the different poles of the first and the second magnets are separated in longitudinal direction. With some examples also the magnetic poles of the third magnet are separated in longitudinal direction.

In effect and according to a further example the magnetic poles of all magnets of the magnetic coupling are separated in longitudinal direction. So, a north pole of magnet may face in one of a proximal or distal direction and a south pole may face to the other one of the proximal and distal direction as defined by the elongation of the longitudinally extending barrel.

According to a further example the first magnet and second magnet each comprise magnetic poles of the first type and of the second type. A longitudinal end of the first magnet that faces towards the second magnet as well as a longitudinal end of the second magnet facing towards the first magnet comprise magnetic poles of a common type. In this way and when the first and second magnets are e.g. aligned coaxially they are in a repulsive configuration. Hence, that 10 pole of the first magnet facing towards the second magnet is of the same type than the pole of the second magnet facing towards the first magnet. Accordingly, and as the first and the second magnets are brought together in longitudinal direction there will be established a respective repulsion between the first and second magnets. Typically, the first magnet and the second magnet are mutually fixed, e.g. they are fixed to a carrier of the drive member, or, with other examples, wherein the first and second magnets are provided on or in the inner coupling member the two magnets may be fixed on or in the stopper of the cartridge.

By way of a repulsive arrangement of first and second magnets, there are generated respective repulsive magnetic fields, in particular between the first and the second magnets, e.g. in the longitudinal direction, by way of which the first and the second magnets are separated from each other. Such magnetic fields are of particular use for a rather strong and/or precise magnetic interaction with the third magnet.

With another example the first and the second magnets may be arranged in a different orientation, wherein they attract each other. Then, the longitudinal end of the first magnet facing towards the second magnet and a longitudinal end of the second magnet facing towards the first magnet comprise magnetic poles of different type. Here, and for instance a north pole of the first magnet may face a south pole of the second magnet or vice versa, the south pole of the first magnet may face a north pole the second magnet. Then, and in such an arrangement the magnetic field of the arrangement of first and second magnets can be increased, thus leading to an increase of the resulting mechanical forces that arise when the third magnet magnetically interacts with the magnetic field of the first and second magnets.

According to a further example a longitudinal size of the gap between the first magnet and the second magnet is smaller than or equal to a longitudinal extent of the third magnet. By varying the longitudinal size of the gap between the first magnet and the second magnet the magnetic response or a magnetic interaction with the third magnet can be precisely adjusted. This may particularly apply when the first and the second magnets are arranged in a repelling configuration, i.e. when a north pole of the first magnet faces a north pole of the second magnet or when a south pole of the first magnet faces a south pole of the second magnet. Typically, with a decrease of the longitudinal size of the gap between repelling first and second magnets the amplitude of the magnetic force between the outer coupling member and the inner coupling member can be increased. With an increase of the longitudinal size of the gap between the first and second magnets a rather homogeneous magnetic force can be provided over a comparatively large range of relative distances between the outer coupling member and the inner coupling member.

Depending on the specific use of the drive mechanism, e.g. being configured to provide a bolus injection or being configured to provide a continuous flow of the medicament the longitudinal size of the gap between the first magnet and the second magnet can be appropriately adjusted or tuned. With a longitudinal gap size between the first magnet and the second magnet in a range of a longitudinal extent of the third magnet a rather homogeneous or even dampened longitudinal force transfer can be provided between the outer coupling member and the inner coupling member.

According to another example a longitudinal size of the gap between the first magnet and the second magnet is larger than or equal to a longitudinal extent of the third magnet. By further increasing the longitudinal size of the gap an even more homogeneous force transfer can be provided between the outer coupling member and the inner coupling member.

According to another example a distance between the longitudinal center of the first magnet and a longitudinal center of the second magnet is smaller than or equal to a longitudinal extent of the third magnet. With this example the longitudinal extent of the third magnet may be further at least slightly larger than the longitudinal extent of at least one of the first magnet and the second magnet. In this way, and depending on the longitudinal size or extent of the first and/or the second magnet a gap with a rather small longitudinal size between the first and second magnets may be realized. By adjusting or modifying the longitudinal distance and/or the distance between the longitudinal centers of the first and the second magnets in view of the longitudinal extent of the third magnet the magnetic coupling between the outer coupling member and the inner coupling member can be adjusted in accordance to predefined demands.

According to a further example a distance between a longitudinal center of the first magnet and a longitudinal center of the second magnet is larger than or equal to a longitudinal extent of the third magnet. Then, the third magnet may be arranged in its entirety between the longitudinal centers of the first and the second magnets, thus allowing to realize a respective and well-defined interaction and hence a respective force transfer between the outer coupling member and the inner coupling member as the outer coupling member is moved relative to the barrel of the medicament container or cartridge.

According to another example the at least one magnet of the outer coupling member comprises a ring magnet. The ring magnet is configured and/or sized to enclose the barrel in a circumferential direction. Hence, an inner circumference, a diameter or cross-section of the ring magnet is only slightly larger than a corresponding outer circumference, an outer diameter or outer cross-section of the barrel of the cartridge or medicament container. Typically, the barrel is of tubular shape and comprises a constant outer diameter as seen in longitudinal direction. This allows for a sliding displacement of the ring magnet relative to the barrel. It is it is of particular benefit, when a remaining annular gap between an inside of the ring magnet and an outside of the sidewall of the barrel is rather small, e.g. below 2 mm, below 1 mm or below 0.5 mm. This way, a radial distance between the ring magnet and the inner coupling member can be reduced to a minimum, thus providing a good magnetic coupling between the outer coupling member and the inner coupling member.

Typically, and when the first and the second magnets are provided on the outer coupling member, both, the first magnet and the second magnet are implemented as a ring magnet. They may be then arranged coaxial or concentric as seen in longitudinal direction.

According to a further example the at least one magnet of the inner coupling member comprises a rod magnet. The rod magnet may comprise a tubular or any other geometric shape. The rod magnet is sized for insertion into the barrel of the medicament container or cartridge.

According to a further example the inner coupling member comprises the third magnet and the third magnet comprises a rod magnet. Here, the outer coupling member comprises the first magnet and the second magnet. Then, the first magnet and the second magnet are each implemented as a ring magnet. The ring magnet and the rod magnet are arranged concentrically. Hence, as the outer coupling member is moved in longitudinal direction relative to the barrel and hence relative to the stopper by a movement of the drive member the magnetic field generated by the arrangement of the first and second magnet starts to electrically couple or to magnetically interact with the magnetic field of the rod magnet, thereby transferring a respective driving force onto the rock connected and hence onto the doctor for moving the same, e.g. in distal direction.

According to another example the inner coupling member is in a longitudinal abutment with a proximal face of the stopper. Alternatively or in addition, the inner coupling member is fastened or fixed to the stopper, or the inner coupling member is integrated into the stopper. Since at least one of the above-mentioned magnets is a component of the inner coupling member this applies correspondingly also to the respective magnet, e.g. to the third magnet. With some examples the inner coupling member is only provided with a single magnet, e.g. with the third magnet. The third magnet may be in direct or indirect longitudinal abutment with a proximal face of the stopper. Such an arrangement is sufficient to exert a distally directed thrust or pressure onto the stopper. With other examples the inner coupling member is fastened to the stopper or is even integrated, e.g. embedded in the stopper. A fastening or an integration of the inner coupling member to or into the stopper is of particular benefit to implement a body directional movement of the stopper, e.g. in distal direction as well as in proximal direction through the magnetic coupling between the outer coupling member and the inner coupling member.

According to a further example the inner coupling member comprises the first magnet and the second magnet. The first magnet and the second magnet each comprise a rod magnet coaxially arranged and fastened on the inner coupling member. Also here, the first magnet and the second magnet are arranged at a longitudinal distance from each other, i.e. in order to form a gap of predefined longitudinal size between the first magnet and the second magnet. With this example the third magnet may be provided on the outer coupling member and may comprise a ring magnet. The ring magnet and the first and the second rod magnets are then arranged coaxial and/or concentric with regards to an axis of symmetry of the tubular shaped barrel of the medicament container or cartridge. With this example the outer coupling member may comprise only one magnet, which is typically implemented as a ring magnet.

According to another example the outer coupling member comprises the first magnet and the second magnet. The first magnet and the second magnet each comprise a ring magnet coaxially arranged relative to each other and fastened on a carrier or connected to or integrated into the drive member and movable relative to the barrel. With such an implementation the third magnet may be implemented as a rod magnet and may be arranged inside the barrel so as to mechanically engage with the stopper.

According to another example it is the other one of the outer coupling member and the coupling member that comprises the third magnet and a fourth magnet separated from each other in longitudinal direction by a gap of predefined longitudinal size. Here, the outer coupling member may comprise the first magnet and the second magnet and the inner coupling member may comprise the third magnet and the fourth magnet. With this example the first magnet and the second magnet may be implemented as ring magnets. The third magnet and the fourth magnet may be each implemented as rod magnet. The first and the second magnets are mutually fixed to each other so as to define a longitudinal gap therebetween. The same may apply to the third magnet and the fourth magnet. Also, the third magnet and the fourth magnet may be arranged and fixed relative to each other at a predefined gap of predefined longitudinal size. With this example the magnetic interaction between the outer coupling member and the inner coupling member can be precisely tuned and defined, namely by the longitudinal size and by the mutual longitudinal arrangement and distance between the first and the second magnets and between the third and the fourth magnet.

According to a further example the third magnet and the fourth magnet each comprise magnetic poles of a first type and of a second type. A longitudinal end of the third magnet facing towards the fourth magnet and a longitudinal end of the fourth magnet facing towards the third magnet comprise magnetic poles of a common type or of different type. When opposite and mutually facing magnetic poles are of a common type, the third and the fourth magnets may be arranged in a repelling mode. Hence, the third and the fourth magnets then tend to repel each other. In the other configuration, wherein the longitudinal end faces of the third and fourth magnets that face towards each other comprise magnetic poles of different types an attractive interaction is obtained between the third magnet and the fourth magnet. In the repelling mode there are formed two extra poles with a strong magnetic field in the center or in the region between the third and the fourth magnet.

Typically and when the respective magnetic poles that face towards each other are of different type there will be used a spacer between the respective magnets in order to prevent a mutually induced movement towards each other.

According to another example the drive mechanism comprises an electric drive mechanically engaged with a spindle gear. The spindle gear comprises an elongated rod extending in the longitudinal direction. By activating the electric drive either the electric drive or the spindle gear is subject to a movement along the elongation of the elongated rod. Here, the elongated rod may be fixed to a housing of the drive member or to a housing of a respective drug delivery device. With this example the drive member may be mechanically fixed to the electric drive or to the spindle gear. Activation of the drive will lead to a respective movement of the drive member relative to the elongated rod in longitudinal direction of the elongated rod. Typically, the elongated rod is a threaded rod.

With other examples the drive member may be mechanically connected to the elongated rod. It may comprise a nut in threaded engagement with the threaded elongated rod. A rotation of the elongated rod, e.g. induced by the activation of the electric drive may lead to a longitudinal displacement of the drive member due to the threaded engagement of the drive member and the elongated rod.

Here, the drive member may be splined to the drive mechanism or to a housing of the drug delivery device such that the drive member is allowed to move in longitudinal direction but is hindered to rotate relative to the rotation axis of the elongated rod.

With another example the drive mechanism comprises an array or a regular arrangement of electromagnets. There may be provided numerous electromagnets arranged adjacently in longitudinal direction, such that the array of electromagnets extends in the longitudinal direction. Here, the electromagnets are controlled by an electronic control and can be individually activated in order to magnetically interact with at least one magnet of the outer coupling member for moving the outer coupling member in longitudinal direction.

In effect, the array of electro magnets may enclose the outer coupling member and may provide a kind of a linear drive for the outer coupling member. By appropriately energizing the electromagnets a resulting magnetic field is operable to move the outer coupling member to in the longitudinal direction.

In another aspect the present disclosure relates to a drug delivery device for dispensing a liquid medicament. The drug delivery device comprises a housing configured to hold and/or to receive a cartridge. Typically, the cartridge is filled with a liquid medicament and is sealed by a stopper. Typically, the stopper seals a proximal longitudinal end of the cartridge and is movable relative to the cartridge or relative to a tubular-shaped barrel of the cartridge. The drug delivery device further comprises a drive mechanism as described above. The drive mechanism may be integrated into the drug delivery device. It may be arranged and fixed inside the housing of the drug delivery device. Here, the drive member of the drive mechanism may be arranged to move along an accommodation space configured to hold or to accommodate the cartridge inside the housing of the drug delivery device. This way, and when a cartridge is appropriately assembled or arranged inside the housing the drive member and in particular the outer coupling member can slide along the outer circumference of the tubular-shaped cartridge in longitudinal direction of the cartridge.

According to a further example the drug delivery device further comprises a cartridge which is filled with a liquid medicament and which is arranged inside or fastened to the housing of the drug delivery device.

With some examples the drug delivery device comprises an injection device, such as a hand-held injection device. With other examples the drug delivery device comprises an infusion device, e.g. an infusion pump. With other examples the drug delivery device may comprise an inhaler.

Generally and with the drive mechanism as described above the magnetic coupling between the outer coupling member and the inner coupling member provides a rather constant force that can be maintained over a comparatively large relative distance between the outer coupling member and the inner coupling member as the outer coupling member is subject to a longitudinal movement relative to the inner coupling member.

By having at least a first magnet and a second magnet on one of the outer coupling member and the inner coupling member and by keeping these two magnets in a predefined longitudinal distance from each other there can be transferred a comparatively high magnetic force between the arrangement of the first and second magnets with a third magnet.

The magnetic coupling between the outer coupling member and the inner coupling member allows to realize a comparatively short overall longitudinal extension of the drive mechanism. Hence, an elongated piston rod extending in proximal extension of the longitudinally extending cartridge is no longer required. In effect, the longitudinal size of the housing of the drive mechanism or of the housing of the drug delivery device might be predominately defined by the longitudinal extent of the cartridge.

Moreover, the implementation of a first magnet and a second magnet on one of the outer coupling member and the inner coupling member is of particular use for reusable drug delivery devices. Here and especially when the third magnet is located longitudinally between or is longitudinally overlapping with the first magnet and the second magnet there can be provided repulsive and attractive forces between the magnets so as to enable a bidirectional force transfer, e.g. in proximal direction as well as in distal direction from the outer coupling member towards the inner coupling member.

In addition, there can be provided a maximum force in both opposite longitudinal directions. Further, and in addition and compared to preloaded force generating solutions the magnetic coupling can be transferred into a rather force-free initial configuration or storage configuration, in which there is effectively no magnetic interaction between the outer coupling member and the inner coupling member.

The magnets as described herein may be implemented as permanent magnets. They may comprise or may be composed of, for example, at least one of the following materials comprising sintered Neodymium-Iron-Boron (NdFeB), preferably with a medical-grade coating, Samarium-Cobalt (SmCo) and Aluminium-Nickel-Cobalt (AlNiCo).

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term _{“}derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 shows a perspective and cross-sectional view of one example of a drive mechanism of a drug delivery device for delivering a liquid medicament,

FIG. 2 is a schematic illustration of the magnetic coupling in a first configuration,

FIG. 3 schematically shows the magnetic coupling of FIG. 2 in another configuration,

FIG. 4 shows one example of first and second magnets separated in longitudinal direction,

FIG. 5 shows a further example of first and second magnets arranged at a longitudinal distance from each other,

FIG. 6 shows a sequence of five positions of the outer coupling member relative to the inner coupling member in the course of moving the outer coupling member relative to the inner coupling member in distal direction,

FIG. 7 shows numerous diagrams of magnetic forces between the outer coupling member and the inner coupling member in accordance to the five positions of FIG. 6 for three different longitudinal spacings between the first and second magnets,

FIG. 8 is illustrative of two further examples of an arrangement of third and fourth rod magnets, and

FIG. 9 shows another implementation of the drive mechanism featuring numerous electromagnets for inducing a longitudinal movement of the outer coupling member.

### DETAILED DESCRIPTION

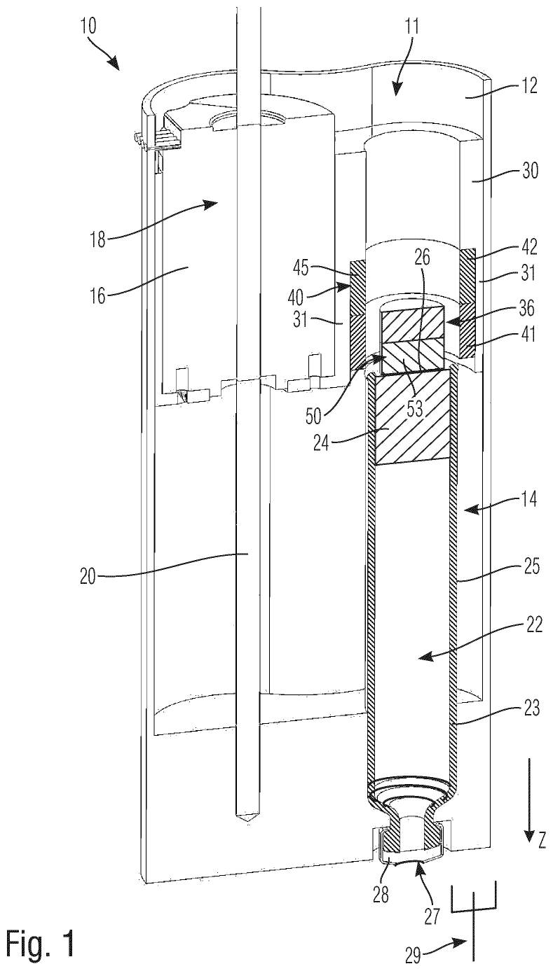

The drug delivery device 10 as illustrated in FIG. 1 comprises a drive mechanism 11 and a housing 12. The drive mechanism 11 is integrated into or arranged inside the housing 12. The housing 12 comprises a compartment 14 sized and configured to receive and/or to hold, i.e. to fix a medicament container 22, which in the present case is implemented as a tubular-shaped cartridge. The cartridge may comprise a vitreous body. The medicament container 22 comprises a tubular-shaped barrel 23, e.g. of vitreous material. The container 22 is sealed in proximal longitudinal direction by a stopper 24. The stopper 24, typically made of an elastomeric material is movable in longitudinal direction inside the tubular-shaped barrel 23. The interior volume of the cartridge is confined by the barrel 23, the stopper 24 and an outlet 27, which is provided at the distal end of the cartridge. The cartridge is fillable or is filled with a liquid medicament 21. The outlet 27 of the cartridge is typically covered or closed by a seal 28. The seal 28 may comprise a rubber disc pierceable by an injection needle 29 in order to expel a dose of the liquid medicament 21 from the interior of the cartridge by displacing or urging the stopper 24 towards the outlet 27.

The outlet 27 of the medicament container 22 may be connected to an injection needle 29, which is only schematically illustrated in FIG. 1. With other examples the outlet 27 may comprise a standardized connector, such as a Luer-type connector way of which an infusion line could be connected with the interior of the medicament container 22 in a fluid transferring manner.

With the presently illustrated example the medicament container 22 is fixed inside the housing 12. It may be detachably fixed inside the housing. Hence, the housing may comprise some kind of a detachable fastener or movable closure by way of which there can be provided access to the interior of the compartment 14 so as to replace the medicament container 22 when empty.

The drive mechanism 11 further comprises an elongated rod 20. The elongated rod 20 extends substantially parallel to the longitudinal direction of the tubular-shaped medicament container 22. The elongated rod 20 may comprise a threaded rod. It may comprise an outer thread. The rod 20 may be in threaded engagement with a spindle gear 18, which in turn is operably engaged with an electric drive 16. As illustrated in FIG. 1 the spindle gear 80 may be located inside a gearbox, e.g. also enclosing the electric drive 16. By activating the electric drive 16, spindle gear 18 also rotates so as to advance along the longitudinal extent of the elongated rod 20. Apparently, the arrangement of the electric drive 16 and the spindle gear 18 is hindered to rotate inside the housing 12. Activation of the electric drive 16 and hence of the spindle gear 80 therefore leads to a longitudinal movement of the electric drive 16 along the elongated rod due to the threaded engagement between the spindle gear 18 and the threaded rod 20.

The drive mechanism 11 further comprises a drive member 30. As illustrated in FIG. 1, the drive member 30 protrudes radially from the elongated rod 24 and is fixedly attached to the electric drive 16 or to the spindle gear 18. Activation of the electric drive 16 leads to a longitudinal sliding motion of the drive member 30. As illustrated, the drive member 30 comprises a kind of a tubular or sleeve-like shape. An inside diameter or cross-section of the drive member 30 is sized to enclose an outside circumference or outside surface 25 of the barrel 23 of the medicament container 22. The drive member 30 further comprises an outer coupling member 40 of a magnetic coupling 36. The magnetic coupling 36 further comprises an inner coupling member 50. The inner coupling member 50 may comprise at least a magnet 53 that is attached to or is at least in longitudinal abutment with a proximal face 26 of the stopper 24.

The inner coupling member 50 is sized and configured for insertion into the proximal end of the barrel 23 of the medicament container 22. The outer coupling member 40 comprises a first magnet 41 and a second magnet 42. Both, the first magnet 41 and the second magnet 42 are implemented as a ring magnet 45. Even though not particularly illustrated in FIG. 1, the first magnet 41 and the second magnet 42 are separated in longitudinal direction ‘(z) by a gap 43 of predefined size. The gap 43 is particularly illustrated in FIGS. 4 and 5.

Also, the ring magnets 45 and hence the first and the second magnets 41, 42 are sized and configured to enclose the outside circumference 25 of the barrel 23. This way, the outer coupling member 40 with the first and the second magnets 41, 42 is configured to slide along the sidewall of the barrel 23.

As further illustrated in FIG. 1 the drive member 30 provides or comprise a carrier 31 for the first magnet 41 and for the second magnet 42, respectively. Hence, the first magnet 41 and the second magnet 42 are attached and/or arranged on or inside the carrier 31 and hence on or inside the drive member 30. The drive member 30 and the carrier 31 may be unitarily formed. With some examples the carrier 31 may be provided by the drive member 30.

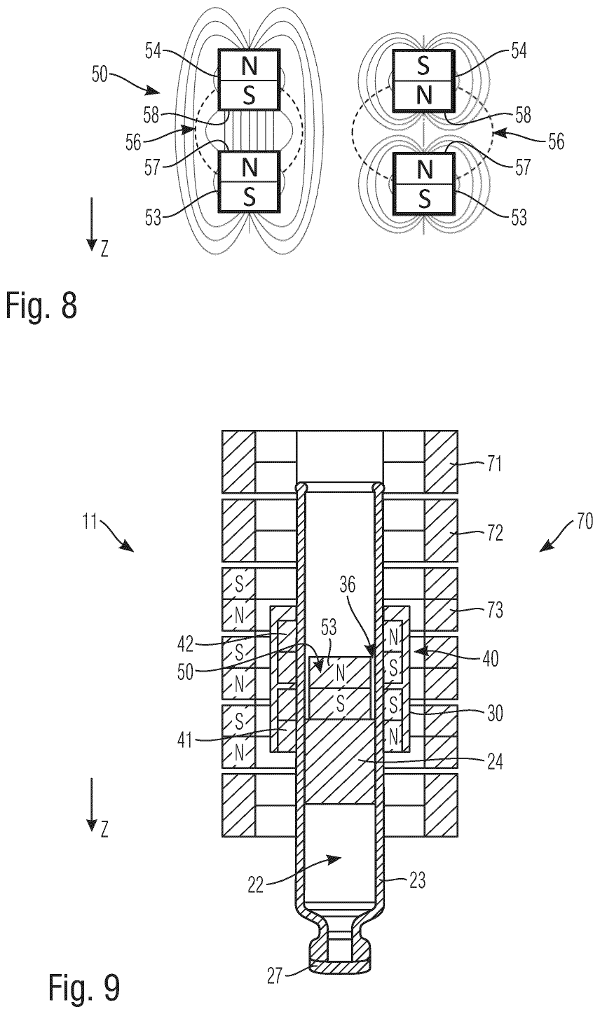

The inner coupling member 50 may comprise a respective carrier (not illustrated). However and as presently illustrated the inner coupling member 50 can be entirely represented or provided by a single magnet 53. With other examples the inner coupling member may be equipped with the third magnet 53 and with an optional fourth magnet 54 as illustrated in FIG. 8. Here, the coupling member 50 comprises a kind of a carrier to which the third and the fourth magnet 34, 54 are mechanically attached or fixed. With further examples the carrier of the inner coupling member 50 may be provided by the stopper 24 of the medicament container 22.

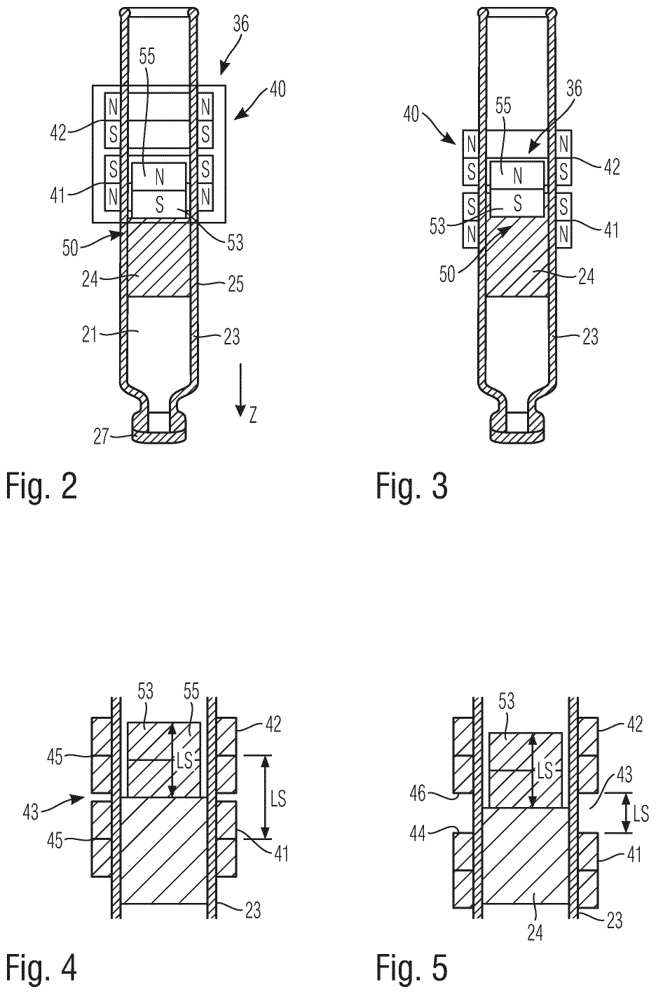

With the Example of FIGS. 1-6, the magnet 53 is a third magnet. It may be the only magnet of the inner coupling member 50. As it is particularly illustrated in FIG. 2 the outer coupling member 40 is slidable relative to the barrel 23 of the medicament container 22 while the third magnet 53, e.g. implemented as a rod magnet 55 is attached to or fixed on the stopper 24 of the medicament container 22. In the configuration of FIG. 2 the outer coupling member 40 has moved a certain distance from a proximal end of the medicament container 22 towards the distal end of the medicament container 22. In the configuration as illustrated in FIG. 2 the first magnet 41 is in a somewhat longitudinal alignment or longitudinal overlaps with the third magnet 53. Here, respective opposite poles of the first magnet 41 and of the third magnet 53 may initially repel. As soon as a configuration as illustrated in FIG. 2 is reached, the respective differently poled sections of the first magnet 41 and of the third magnet 53 may mutually attract.

As the outer coupling member 40 is moved further in distal direction, hence closer towards the outlet 27, as e.g. illustrated in FIG. 3, the interaction between the outer coupling member 40 and the inner coupling member 50 may be further influenced by the magnetic field of the second magnet 42 of the outer coupling member 40. The south pole S of the third magnet 53 may be in repelling interaction with the south pole S of the first magnet 41. The north pole N of the third magnet 53 is in attractive interaction with the south pole P of the second magnet 42.

Experiments a have revealed, that the configuration according to FIG. 2 is a rather powerless configuration, where the longitudinal force effect as transferred from the outer coupling member 40 onto the inner coupling member 50 is at a minimum. In the configuration of FIG. 3, a longitudinal force transfer between the outer coupling member 40 and the inner coupling member 50 is at a maximum.

In FIG. 4 a longitudinal distance LS between a longitudinal center of the first magnet 41 and the longitudinal center of the second magnet 42 is substantially equal to the longitudinal extent or longitudinal size LS of the third magnet 53.

In FIG. 5, a different size of a gap 43 is provided. There, the longitudinal size LS of the gap 43 between the first magnet 41 and the second magnet 42 is substantially equal to the longitudinal size or longitudinal extent of the third magnet 53. Here, and by tuning the longitudinal size of the individual magnets 41, 42, 53 and further by varying the longitudinal distance between the first magnet 41 and the second magnet 42 the force transfer between the outer coupling member 40 and the inner coupling member 50 can be appropriately modified, e.g. in accordance to the relevant application scenario.

With the example of FIGS. 2-7 the first magnet 41 and the second magnet 42 are arranged in a repelling configuration. Hence, a longitudinal end 44 of the first magnet 41 facing towards the second magnet 42 and the longitudinal end 46 of the second magnet 42 facing towards the first magnet 41 comprise the same polarity or comprise the same magnetic pole. With the example of FIGS. 2 and 3 the south poles of the first magnet 41 and the second magnet 42 are facing towards each other.

Consequently, there will be provided a comparatively strong magnetic field with two extra magnetic poles in the center or in the region between the first and the second magnets 41, 42. These supplemental magnetic poles, as e.g. illustrated in FIG. 8, are of particular benefit to realize a comparatively strong longitudinal force transfer between the outer coupling member 40 and the inner coupling member 50.

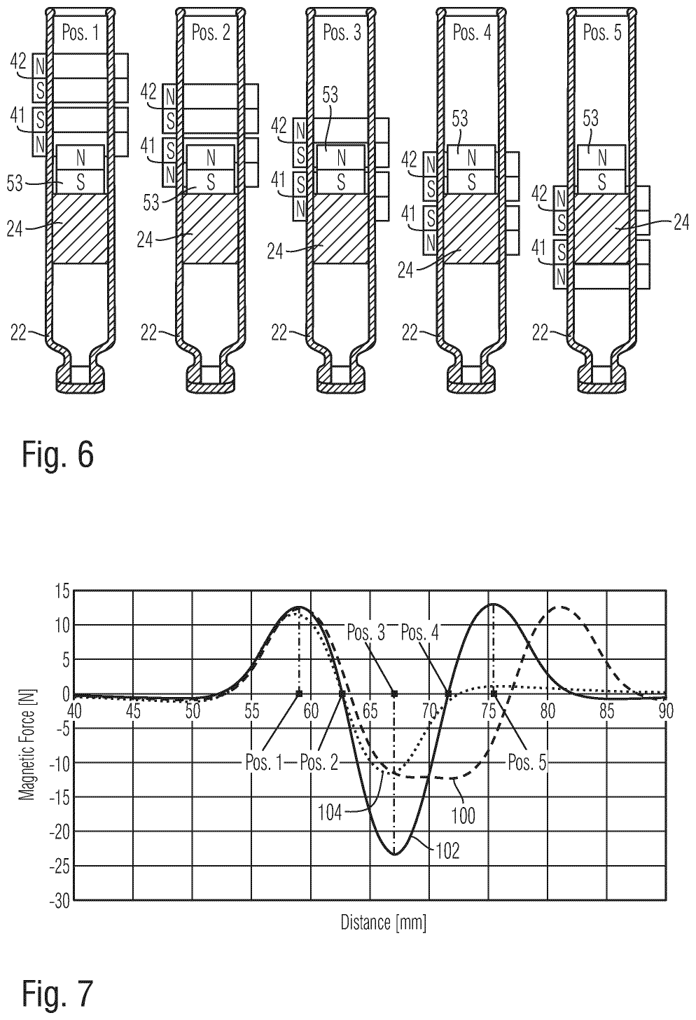

In the illustration of FIG. 6 the outer coupling member 40 is illustrated in five different positions Pos. 1, Pos. 2, Pos. 3, Pos. 4, and Pos. 5 as it is moved by the drive member 30 from a proximal end position as shown in position Pos. 1 towards a distal end position Pos. 5. The respective positions are also reflected in the graph of FIG. 7. Moreover, in FIG. 7 there are illustrated three 3 different measured curves 100, 102, 104 of a magnetic force transfer versus the displacement of the outer coupling member 40 relative to the medicament container 22. Here, the curve 104 is a comparative example showing a force transfer versus the longitudinal displacement when the outer coupling member 40 only comprises a single ring magnet 45.

The curve 100 represents an example, wherein a comparatively large longitudinal spacing between the first magnet 41 and the second magnet 42 is realized. Here, the longitudinal size of a gap 43 between the first magnet 41 and the second magnet 42 was about 8 millimeters. The longitudinal size of the gap 43 may be then in a region of the longitudinal extent of the third magnet 53.

As it is immediately apparent from the curve 100, its lower peak relating to Pos. 3 and Pos. 4 has significantly flattened. There is hence provided a rather constant force over a comparatively long relative distance. This is of particular benefit to provide a constant longitudinal force transfer irrespective of a longitudinal displacement of the outer coupling member 40 relative to the medicament container 22. With the further curve 102, the distance and hence the gap size between the first and the second magnets 41, 42 has been reduced, e.g. to a size of about 2 mm. The longitudinal size of the gap may be smaller than 50%, smaller than 30% or smaller than 20% of the longitudinal extent of the third magnet 53.

As it is immediately apparent, the amplitude of the magnetic force transfer substantially increases compared to the amplitude of the comparative curve 104. Hence, with this configuration and with the relative position at Pos. 3, which is also reflected in FIG. 3, there can be provided a longitudinal force effect or force transfer that is about twice as large as compared to a solution with only one ring magnet 45.

As the outer coupling member 40 is moved towards and into Pos. 1 as illustrated in FIGS. 6 and 7 there is initially applied a distally directed repelling force onto the third magnet 53 thus urging the third magnet 53 in distal direction.

In Pos. 2 there is no net longitudinal force effect. As the outer coupling member 40 is moved further in distal direction there is applied a distally directed force onto the magnet 53. Here, a maximum force transfer between the outer coupling member 30 and the inner coupling member 50 for moving the inner coupling member 50 and hence the stopper 24 is provided.

In the configuration of FIG. 3 and due to the rather symmetric arrangement of the first and the second magnets 41, 42 as seen in longitudinal direction (z) it is also possible to move the third magnet 53 in the opposite direction, hence towards the proximal end, namely by moving the outer coupling member 40 in the opposite, hence proximal direction, i.e. away from the outlet 27.

In FIG. 8 there are illustrated two further examples of the inner coupling member 50 comprising not only a third magnet 53 but also comprising a fourth magnet 54. Here, the third magnet 53 and the fourth magnet 54 are both implemented as rod magnet 55. They are arranged coaxial and are separated along the longitudinal direction (z) by a gap 56 of predefined longitudinal size.

With the example as shown to the left in FIG. 8 the third magnet 53 and the fourth magnet are co-aligned in an attracting configuration. Here, a longitudinal end 57 of the third magnet 53 facing towards the fourth magnet 54 and a longitudinal and 58 of the fourth magnet 54 facing towards the third magnet 53 comprise magnetic poles of different type. Accordingly, the third magnet 53 and the fourth magnet 54 attract each other.

Accordingly, the resulting magnetic field is lower than that of oppositely oriented rod magnets as shown in the right-handed example of FIG. 8. There, the longitudinal end 57 of the third magnet 53 facing towards the fourth magnet 54 and the longitudinal end 58 of the fourth magnet 54 facing towards the third magnet 53 comprise magnetic poles of a common type.

Accordingly, there are formed two extract magnetic poles with a strong magnetic field in the center, which may interact with the respective resulting magnetic fields of the first and the second magnets 41, 42.

In FIG. 9, another example of a drive mechanism 11 is illustrated. Instead of an electric drive 16 and a spindle gear 18 as illustrated in FIG. 1 the drive mechanism 11 comprises a number of individual electromagnets 71, 72, 73. Each electromagnet 71, 72, 73 typically comprises a coil and so as to generate a magnetic field when subject to a respective current flow. The individual electromagnets 71, 72, 73 form or constituted an array 70 of electromagnets extending in the longitudinal direction (z) and hence parallel to the elongation of the medicament container 22.

The outer coupling member 40 comprises a first ring magnet 41 and a second ring magnet 42 that are arranged concentrically and in a longitudinal distance from each other. The electromagnets 71, 72, 73 may be implemented as so-called air coils and may be covered with a sheet of a so-called μ-metal to ensure a sufficient magnetic flux. By way of a controller (not illustrated) the individual coils and hence electromagnets 71, 72, 73 can be energized so as to induce a longitudinal movement of the outer coupling member 40 relative to the barrel 23. Here, the first and second magnets 41, 42 of the outer coupling member 40 provide and fulfill a double function.

By way of a first magnetic coupling with the array 70 of electromagnets 71, 72, 73 the outer coupling member 40 can be moved in longitudinal direction (z) relative to the medicament container 22. By way of the magnetic coupling 36 between the first and second magnets 41, 42 of the outer coupling member 40 with the third magnet 53 of the inner coupling member 50 the longitudinal movement of the outer coupling member 40 can be transferred into a respective longitudinal advancing or displacement of the stopper 24.

### Reference Numbers

10 drug delivery device
11 drive mechanism
12 housing
14 compartment
16 electric drive
18 spindle gear
20 rod
21 medicament
22 medicament container
23 barrel
24 stopper
25 outside surface
26 proximal face
27 outlet
28 seal
29 injection needle
30 drive member
31 carrier
36 magnetic coupling
40 outer coupling member
41 magnet
42 magnet
43 gap
44 longitudinal end
45 ring magnet
46 longitudinal end
50 inner coupling member
53 magnet
54 magnet
55 rod magnet
56 gap
57 longitudinal end
58 longitudinal end
70 array
71 electromagnet
72 electromagnet
73 electromagnet

1-19. (canceled)

20. A drive mechanism for moving a stopper of a medicament container, wherein the stopper is movably disposed inside a longitudinally extending barrel of the medicament container, the drive mechanism comprising:
a drive member movable along a longitudinal direction (z) parallel to the longitudinal extent of the barrel,
a magnetic coupling comprising an outer coupling member and an inner coupling member, wherein the outer coupling member, which is mechanically engaged with the drive member, is configured to move along an outside surface of the barrel and wherein the inner coupling member, which is mechanically engagable with the stopper, is configured for insertion into the barrel,
wherein at least one of the outer coupling member and the inner coupling member comprises a first magnet and a second magnet separated from each other in the longitudinal direction (z) and being configured to magnetically couple with a third magnet of the other one of the outer coupling member and the inner coupling member.

21. The drive mechanism according to claim 20, wherein the first magnet and the second magnet are separated by a gap of predefined longitudinal size.

22. The drive mechanism according to claim 21, wherein a longitudinal size of the gap between the first magnet and the second magnet is smaller than or equal to a longitudinal extent of the third magnet.

23. The drive mechanism according to claim 21, wherein a distance between a longitudinal center of the first magnet and a longitudinal center of the second magnet is smaller than or equal to a longitudinal extent of the third magnet.

24. The drive mechanism according to claim 20, wherein at least one of the first magnet and the second magnet comprises a magnetic pole of a first type (N) and another magnetic pole of a second type(S), the magnetic poles of the first and second type (N, S) being separated in longitudinal direction (z).

25. The drive mechanism according to claim 24, wherein the first magnet and the second magnet each comprise magnetic poles of the first type (N) and of the second type(S), wherein a longitudinal end of the first magnet facing towards the second magnet and a longitudinal end of the second magnet facing towards the first magnet comprise magnetic poles (N, S) of a common type.

26. The drive mechanism according to claim 20, wherein the at least one magnet of the outer coupling member comprises a ring magnet configured and/or sized to enclose the barrel in a circumferential direction.

27. The drive mechanism according to claim 20, wherein the at least one magnet of the inner coupling member comprises a rod magnet.

28. The drive mechanism according to claim 20, wherein the inner coupling member comprises the third magnet and wherein the third magnet comprises a rod magnet.

29. The drive mechanism according to claim 20, wherein the inner coupling member is one of:
in a longitudinal abutment with a proximal face, of the stopper,
fastened to the stopper, or
integrated into the stopper.

30. The drive mechanism according to claim 20, wherein the inner coupling member comprises the first magnet and the second magnet, the first magnet and the second magnet each comprise a rod magnet coaxially arranged and fastened on the inner coupling member.

31. The drive mechanism according to claim 20, wherein the outer coupling member comprises the first magnet and the second magnet, the first magnet and the second magnet each comprise a ring magnet coaxially arranged and fastened on a carrier connected to or integrated into the drive member movable relative to the barrel.

32. The drive mechanism according to claim 20, wherein the other one of the outer coupling member and the inner coupling member comprises the third magnet and a fourth magnet, separated from each other in longitudinal direction (z) by a gap of predefined longitudinal size.

33. The drive mechanism according to claim 20, wherein the third magnet and the fourth magnet, each comprise magnetic poles of the first type (N) and of the second type(S), wherein a longitudinal end of the third magnet facing towards the fourth magnet, and a longitudinal end of the fourth magnet, facing towards the third magnet comprise magnetic poles (N, S) of a common type or of different type.

34. The drive mechanism according to claim 20, wherein the drive member comprises an electric drive mechanically engaged with a spindle gear, wherein the spindle gear comprises an elongated rod extending in the longitudinal direction (z).

35. The drive mechanism according to claim 20, further comprising an array of electromagnets extending in the longitudinal direction (z) and operable to magnetically interact with the at least one magnet of the outer coupling member for moving the outer coupling member in longitudinal direction (z).

36. The drive mechanism according to claim 20, wherein the outer coupling member is mechanically connected or fixed to the drive member and is movable along the outside surface of the barrel.

37. A drug delivery device for dispensing a liquid medicament, the drug delivery device comprising:
a housing configured to hold and/or to receive a medicament container filled with the liquid medicament and sealed by a stopper, and
a drive mechanism according to claim 20.

38. The drug delivery device according to claim 37, further comprising the medicament container arranged inside or fastened to the housing.
